(19) United States
(12) Patent Application Publication
(10) Pub. No.: US 2017/0086792 A1
(43) Pub. Date: Mar. 30, 2017

(54) ULTRASONIC DIAGNOSTIC DEVICE

(71) Applicant: Hitachi, Ltd., Tokyo (JP)

(72) Inventor: Tomoaki CHONO, Tokyo (JP)

(73) Assignee: Hitachi, Ltd., Tokyo (JP)

(21) Appl. No.: 15/126,471

(22) PCT Filed: Nov. 5, 2014
(86) PCT No.: PCT/JP2014/079345

§ 371 Date: Sep. 15, 2016

(30) Foreign Application Priority Data
Mar. 18, 2014 (JP) 2014-055559

(51) Int. Cl. A61B 8/08; A61B 8/00; A61B 8/06

(52) U.S. Cl. CPC A61B 8/488; A61B 8/065; A61B 8/463; A61B 8/469; A61B 8/4245; A61B 8/5269

(57) ABSTRACT

Provided is an ultrasonic diagnostic device in which a Doppler waveform generation unit performs Doppler measurement at a set Doppler measurement position and generates a current Doppler waveform. A Doppler waveform evaluation unit evaluates the generated current Doppler waveform and determines whether the current Doppler waveform is a suitable Doppler waveform at the target measurement position. When it is determined that the current Doppler waveform is not a suitable waveform at the target measurement position, a Doppler measurement position variation unit performs processing for varying the Doppler measurement position. The Doppler measurement position is varied continuously until the post-variation Doppler measurement position becomes the target measurement position.

### TECHNICAL FIELD

The present invention relates to an ultrasonic diagnostic device, and more particularly to techniques of shifting a Doppler measurement position to a correct position.

### BACKGROUND

Ultrasonic diagnostic devices transmit and receive ultrasound waves to and from an examinee, and, based on a received signal thus obtained, form an ultrasound image. The ultrasonic diagnostic devices have a Doppler measurement function for detecting the moving direction and the moving velocity of a target subject using a Doppler effect. The Doppler measurement function is used for measuring a blood flow velocity, for example.

Known Doppler measurement methods include, for example, a method called color Doppler, for performing Doppler measurement in a wide range to obtain a color Doppler image that indicates distribution of the velocity of blood flow within the range, a method called continuous wave Doppler, for performing Doppler measurement using continuous waves over a wide range of an ultrasound beam, and a method called pulsed Doppler for performing Doppler measurement using pulse waves in a local area of an ultrasound beam. All of these methods require appropriate setting of a measurement range or a measurement position. A user moves area frames, lines, and cursors on the screen with a track ball or the like provided on an operation panel to thereby specify the measurement range or the measurement position.

In the continuous wave Doppler and the pulsed Doppler, blood flow motion information is measured by transmitting and receiving ultrasound waves to and from a designated measurement position, which may be also referred to as a range gate, sample volume, sample gate, for example, and frequency analysis of the blood flow motion information is used to generate Doppler waveforms. In the Doppler waveforms, the horizontal axis indicates a time axis and the vertical axis is a frequency axis, i.e., a velocity axis. A Doppler waveform is a waveform which represents, as a luminance value, the power of each frequency component constituting a spectrum as a result of the frequency analysis.

In some instances, the measurement position may be displaced from the position where a user originally wished to perform measurement due to a movement of the examinee, vibrations of a user's hand holding the probe, and the like. This may result in a change in the shape of the Doppler waveforms, including a reduction in the size of the Doppler waveform in the vertical axis direction, for example, and may even result in disappearance of the Doppler waveforms. To deal with such a situation, a user must perform an operation for resetting the measurement position to the target measurement position manually using an input device, such as a track ball. Resetting the measurement position requires complicated operations to be performed by the user.

Techniques for automatically correcting the Doppler measurement position have been proposed. Patent Document 1 discloses a technique for setting a large number of Doppler measurement positions, i.e., range gate positions, near the current Doppler measurement position, and setting the center of a position where it is determined that blood flow exists, based on a color Doppler tomographic image including blood flow information, to a new Doppler measurement position.

### CITATION LIST

### Patent Literature

### [Patent Document 1] JP 6-217975 A

### SUMMARY

### Technical Problem

As described above, when the Doppler measurement position has been displaced to an inappropriate position due to extraneous disturbance factors, it is not possible to obtain suitable Doppler waveforms. If a user, e.g., a doctor or a clinical examination technician, cannot recognize this situation, there arises a hindrance to diagnosis. Even if the user can recognize the problem, resetting the Doppler measurement position to the originally set position would require complicated operations. On the other hand, it is necessary to set the Doppler measurement position precisely to a portion corresponding to a diagnosis target, e.g., mitral valve outflow side, which generally requires time and skill. As such, a reduction in the burden placed on users for setting the Doppler measurement position is desired.

In the disclosure of Patent Document 1, because a new Doppler measurement position is set to a position where blood flow exists, there is no guarantee that the Doppler waveforms in the new Doppler measurement position will be suitable waveforms in the target measurement position, and whether or not the Doppler waveforms in the new Doppler measurement position will be effective as diagnosis data is not guaranteed, for example.

An advantage of the invention is to provide an ultrasonic diagnostic device which is capable of shifting a Doppler measurement position to a target measurement position. Another advantage is to reduce the user's burden in the Doppler measurement. A further advantage is to make the result of the Doppler measurement useful in Doppler measurement control.

### Solution to Problem

In accordance with one aspect, an ultrasonic diagnostic device includes a Doppler waveform generator configured to generate, based on a received signal obtained by transmitting and receiving ultrasound waves to and from a Doppler measurement position set for measuring a motion concerning a target tissue in a target measurement position within the target tissue, a Doppler waveform indicating the motion in the Doppler measurement position, a waveform evaluator configured to perform waveform evaluation of the Doppler waveform, to thereby evaluate whether or not the Doppler waveform satisfies a suitable condition required for a suitable Doppler waveform in the target measurement position, and a Doppler measurement position shifter configured to shift the Doppler measurement position when the Doppler waveform does not satisfy the suitable condition.

In some embodiments, the Doppler measurement position shifter may shift the Doppler measurement position to a measurement position where a Doppler waveform which satisfies the above suitable condition can be obtained. In some embodiments, the suitable condition may be a condition concerning a degree of correspondence between a current Doppler waveform generated by the Doppler waveform generator and a reference waveform to be compared therewith, and the waveform evaluator may determine whether or not the suitable condition is satisfied based on the degree of correspondence. In some embodiment, the waveform evaluator may calculate, as the degree of correspondence, a degree of correspondence of waveform shapes, based on a shape of the reference waveform and a shape of the current Doppler waveform. In some embodiments, the waveform evaluator may calculate, as the degree of correspondence, a degree of correspondence of parameters, based on a parameter representing a feature of the reference waveform and a parameter representing a feature of the current Doppler waveform.

The Doppler waveform generator performs Doppler measurement at a Doppler measurement position which is manually set by a user, for example, to generate a Doppler waveform representing a distribution of blood flow velocity, i.e., spectrum, at the Doppler measurement position. On the other hand, the waveform evaluator determines whether or not the Doppler waveform generated by the Doppler waveform generator is suitable as a Doppler waveform in the target measurement position based on waveform evaluation. It is desirable for the determination to be executed in synchronism with the generation of a waveform, i.e., in real time. If the Doppler waveform set in the Doppler measurement position is suitable, the set Doppler measurement position can be determined to be the target measurement position which is originally set or is ideal. The target measurement position can be defined by setting the waveform evaluation method or evaluation criteria as appropriate. For example, whether or not the measured Doppler waveform is suitable can be determined based on the degree of correspondence between the measured Doppler waveform and the reference waveform. Alternatively, comparison between a parameter indicating the features of the measured Doppler waveform, such as a wave height, waveform thickness, for example, and a parameter indicating the features of the reference waveform may be used for determination.

What is performed in this case is also essentially a comparison of the waveforms. If the Doppler waveform which is measured by the waveform evaluator is not suitable as a Doppler waveform in the target measurement position, as this means that the set Doppler measurement position is not the target measurement position, the Doppler measurement position shifter performs a process for shifting the set Doppler measurement position. It is preferable for the shift of the Doppler measurement position to be performed continuously until the Doppler measurement position which is shifted matches the target measurement position.

The Doppler waveform realistically reflects a motion state in the Doppler measurement position, and generally reacts responsively to a displacement of the Doppler measurement position, e.g., a relative displacement with respect to the blood flow. Accordingly, use of such a Doppler waveform as a subject of evaluation enables objective and speedy determination as to whether or not the Doppler measurement is suitable. Methods of evaluating the Doppler waveform include a method of evaluating the characteristic quantity of the waveform itself in an absolute manner, a method of evaluating in a relative manner based on direct or indirect comparison with a reference waveform, and other methods. As the Doppler waveform generally has a complicated shape for which absolute evaluation is difficult, the latter method is desirably adopted. In this case, the reference waveform maybe a Doppler waveform which is sampled when starting the current Doppler measurement, a Doppler waveform which was sampled in the past, a Doppler waveform which is generated by simulation, and the like. It is desirable for the reference waveform to be prepared for each measurement position.

In the above structure, the waveform evaluator may calculate an evaluation value based on the degree of correspondence and indicate the evaluation value on a display unit. The evaluation value may be, for example, an index showing to what degree the current Doppler waveform is suitable as a Doppler waveform in the target measurement position. By displaying the evaluation value on the display unit, it is possible to present to the user an index showing the degree of displacement of the current Doppler measurement position with respect to the target measurement position. The user, confirming the evaluation value, can perform, by themselves, subtle adjustment of the Doppler measurement position, for example, before automatic shift of the Doppler measurement position is started. Display of the evaluation value is especially useful when the automatic shift of the Doppler measurement position is not performed. Display of a graph indicating a change in the evaluation value with time allows prediction of whether or not any correction for positional displacement is necessary based on the transition or tendency of change of the evaluation value.

In the above structure, the waveform evaluator may determine whether or not the Doppler waveform satisfies the suitable condition in a predetermined time phase section during a pulsation cycle of the target tissue. Further, the target tissue may be a heart, and the waveform evaluator may determine the predetermined time phase section based on an electrocardiographic waveform of the heart. Appropriate setting of the time phase section enables adjustment of reliability and responsivity of the waveform evaluation. When the time phase section is determined based on the electrocardiographic waveform, for example, setting a section from an R wave to an R wave, that is, an entire portion of one pulsation cycle, as the subject of waveform evaluation, would allow more accurate calculation of the degree of correspondence between the measured Doppler waveform and the reference waveform, so that reliability of the waveform evaluation can be assured. When only a part of one pulsation cycle is set as the subject of waveform evaluation, on the other hand, while the correctness of the degree of correspondence between the measured Doppler waveform and the reference waveform is lowered because the whole of the waveform in one pulsation cycle is not evaluated, the responsiveness of the waveform evaluation would increase because it is possible to complete the waveform evaluation rapidly. The time phase section may be automatically set in accordance with the target portion of the Doppler measurement, for example.

In the above structure, the Doppler measurement position shifter may search for a new measurement position within a search range determined based on the Doppler measurement position. The search range can be set to an area in the vicinity of the set Doppler measurement position, for example, because even when the set Doppler measurement position is not a target measurement position, the target measurement position often exists in the vicinity of the set Doppler measurement position. By searching for a new Doppler measurement position in the vicinity of the set Doppler measurement, it is possible to set the Doppler measurement position to the target measurement position at an earlier stage.

### Advantageous Effects of Invention

According to the invention, it is possible to shift a Doppler measurement position to a target measurement position. It is further possible to alleviate a burden on the user in the Doppler measurement. It is also possible to allow a result of the Doppler measurement to be utilized in control of the Doppler measurement.

### BRIEF DESCRIPTION OF DRAWINGS

FIG. 1 is a diagram schematically illustrating a structure of an ultrasonic diagnostic device according to an embodiment of the invention.

FIG. 2 is a conceptual view illustrating comparison between the current Doppler waveform and suitable waveform information.

FIG. 3 is a diagram illustrating example waveform parameters.

FIG. 4 is a diagram illustrating an example search range based on the current Doppler measurement position.

FIG. 5 is a diagram illustrating an example search range based on a reference portion of a target tissue.

FIG. 6 is a diagram illustrating an example search range based on blood flow information.

FIG. 7 is a diagram illustrating displacement of a Doppler measurement position from a target measurement position.

FIG. 8 is a diagram illustrating reset of a Doppler measurement position to a target measurement position.

FIG. 9 is a flowchart illustrating a flow of operations by the ultrasonic diagnostic device according to an embodiment.

### DESCRIPTION OF EMBODIMENTS

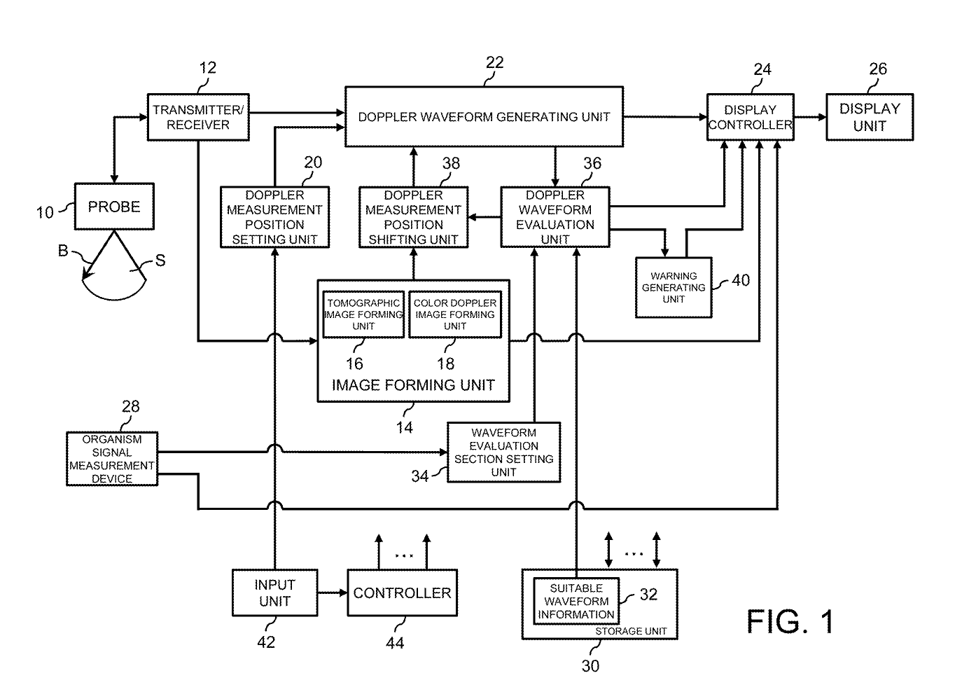

Embodiments of an ultrasonic diagnostic device will be described hereinafter. It should be noted that the present invention is not limited to the following embodiments. FIG. 1 is a diagram schematically illustrating a structure of an ultrasonic diagnostic device according to an embodiment.

A probe 10 is an ultrasound probe which transmits and receives ultrasound waves to and from a target tissue. The target tissue is an organism tissue in which blood flows and, in the present embodiment, is a heart. A blood vessel or other circulatory organ tissues may be the target tissue. The probe 10 includes an array transducer formed of a plurality of transducer elements, and the array transducer forms an ultrasound beam B. Also, a scanning plane S is formed by electronic scanning of the ultrasound beam B. Methods for the electronic scanning include, for example, an electronic sector scanning method and an electronic linear scanning method. The probe 10 may include a 2D array transducer to allow capturing of three-dimensional data. As will be described below, based on a received signal obtained by scanning an ultrasound beam with the probe 10, a tomographic image of a target tissue and a color Doppler image showing a distribution of blood flow within the target tissue are captured. Further, with Doppler observation in a specific orientation and depth, a Doppler waveform representing a change in the velocity spectrum of the blood flow with time, for example, is formed. Known Doppler measurement modes include a PW mode and a CW. Waveform evaluation and the like which will be described below are applicable to either mode.

A transmitter/receiver unit 12 transmits a plurality of transmitting signals, which oscillate a plurality of transducer elements of the probe 10, to the probe 10, thereby causing the probe 10 to generate ultrasound waves. The transmitter/receiver unit 12 also performs phase alignment and summation processing with respect to a plurality of received signals obtained from the plurality of transducer elements of the probe 10, thereby forming a received beam, that is, a received signal, i.e., beam data, having undergone the phase alignment and summation processing. As such, the transmitter/receiver unit 12 has functions of a transmitting beam former and a received beam former.

An image forming unit 14 forms various images based on the received signal supplied from the transmitter/receiver unit 12. The image forming unit 14 includes a tomographic image forming unit 16 and a color Doppler image forming unit 18.

The tomographic image forming unit 16, based on image capturing setting set by a user, e.g., a scanning range of the ultrasound beam and gain setting, forms a tomographic image which is an ultrasound image, from the received signal supplied from the transmitter/receiver unit 12. In the present embodiment, the tomographic image is a B-mode image representing a cross section of the target tissue as an image. A three-dimensional image may be formed based on a plurality of two-dimensional tomographic image data items. The tomographic image is stored in a storage unit 30 and is also displayed on a display unit 26 by a display controller 24.

The color Doppler image forming unit 18, based on the received signal obtained by Doppler measurement performed in an area set by the user, calculates two-dimensional distribution of the velocity of blood flow within the target issue. The color Doppler image forming unit 18 further performs conversion of the velocity to a luminance value, coloring, and the like, based on the calculated velocity distribution, thereby forming a color Doppler image having colors representing the blood flow superposed. The display controller 24, which will be described below, has an image synthesis function, thereby synthesizing the color Doppler image on the tomographic image formed by the tomographic image forming unit 16. Thus, a color flow mapping (CFM) image is formed.

A Doppler measurement position setting unit 20 sets a Doppler measurement position within a target tissue. Ultrasound waves from the probe 10 are transmitted to and received from the Doppler measurement position set by the Doppler measurement position setting unit 20. Based on the received signal thus generated, a Doppler waveform generating unit 22 generates a Doppler waveform representing movement of the blood flow in the Doppler measurement position. The Doppler measurement position setting unit 20, based on an instruction from an input unit 42, sets the Doppler measurement position. For example, the user moves a track ball or the like included in the input unit 42 on a B-mode image displayed on the display unit 26 to move a cursor indicating the Doppler measurement position to a desired position, thereby specifying the Doppler measurement position. The Doppler measurement position setting unit 20 sets a position specified by the input unit 42 as a Doppler measurement position. Alternatively, a Doppler measurement position may be automatically set. For example, a Doppler measurement position may be specified based on a reference portion representing a feature of a shape of the target tissue obtained from the B-mode image or based on the blood flow information provided by a CFM image.

The Doppler waveform generating unit 22 converts each velocity component value to luminance, based on spectrum information obtained by frequency analysis of a received signal obtained by Doppler measurement using pulsed Doppler, continuous wave Doppler, color Doppler, power Doppler, tissue Doppler, and the like, performed in the Doppler measurement position, thereby generating a Doppler waveform representing a velocity distribution of a tissue such as blood flow at the Doppler measurement position. The velocity of a tissue in a Doppler measurement position generally includes a plurality of velocity components or velocity component distribution, and each velocity component is converted to luminance at each time point. In a distribution of the velocity components which are measured, a higher luminance is assigned to a greater flow velocity component. As described above, a Doppler waveform is generated as a waveform having a fixed width at each time and having velocity component values in a belt shape, i.e., a waveform having a thickness. The Doppler waveform is continuously updated, and is stored in the storage unit 30 and simultaneously displayed on the display unit 26 under control of the display controller 24.

The display controller 24 processes signals output from the image forming unit 14, the Doppler waveform generating unit 22, an organism signal measuring device 28 which will be described below, and a warning generating unit 40, and outputs processed data to the display unit 26.

The display unit 26 is a monitor, e.g., CRT and LCD, and displays various images formed by the image forming unit 14, organism signal waveforms obtained by the organism signal measuring device 28, Doppler waveforms generated by the Doppler waveform generating unit 22, and warnings and the like generated by the warning generating unit 40. The display unit 26 further displays a cursor indicating the Doppler measurement position.

The organism signal measurement device 28 receives an organism signal of the target tissue and generates organism signal data. The organism signal data include electrocardiographic waveforms, phonocardiography waveforms, and the like. The organism signal data are transmitted to the display controller 24 and displayed on the display unit 26 and also stored in the storage unit 30.

The storage unit 30 stores therein a tomographic image and a color Doppler image formed by the image forming unit 14, a Doppler measurement position set by the Doppler measurement position setting unit 20, organism signals measured by the organism signal measurement device 28, and Doppler waveforms formed by the Doppler waveform generating unit 22. The storage unit 30 also stores therein programs, calculation operation systems, and estimation operation systems for actuating various functions of the ultrasonic diagnostic device. The storage unit 30 is a storage medium, such as a semiconductor memory, an optical disk, and a magnetic disk, or may be an external storage medium connected via the network.

The storage unit 30 further stores therein suitable waveform information 32 indicating a suitable Doppler waveform. The suitable waveform information 32 is used for comparison with a Doppler waveform obtained by transmission and reception of ultrasound waves to the Doppler measurement position which is currently set, hereinafter referred to as the “current Doppler waveform”, in order to evaluate the current Doppler waveform. Because the suitable Doppler waveform is different among different measurement positions within the target tissue or measurement items, the suitable waveform information 32 is prepared for each measurement position or each measurement item. Further, as the suitable Doppler waveform may also vary depending on an examinee even with respect to the same measurement position and the same measurement item, the suitable waveform information 32 may be prepared for each examinee.

The suitable waveform information 32 may be a Doppler waveform, i.e. a reference waveform, itself. Of course, the reference waveform is an appropriate Doppler waveform. In this case, Doppler waveforms obtained by the Doppler measurement performed in the past are stored, as the reference waveforms, in the storage unit 30, for example. Alternatively, among the Doppler waveforms obtained by the Doppler measurement in the past, only Doppler waveforms designated by the user may be stored as the reference waveforms. Also, an average of the Doppler waveforms obtained by the previous Doppler measurements may be used as the reference waveform, or an ideal Doppler waveform is prepared as the reference waveform. At the time of starting the current Doppler measurement, that is, at the time of setting the measurement position, the Doppler waveform which is obtained may be stored as the suitable waveform information 32.

The suitable waveform information 32 may be a waveform parameter indicating a feature of the Doppler waveform, rather than the Doppler waveform itself. The waveform parameter can be obtained from the Doppler waveforms obtained by Doppler measurement performed in the past, for example. The waveform parameter may include, for example, a wave height, a waveform thickness, an attenuation time, a noise amount, and the like. The waveform parameter may be obtained from a waveform obtained from Doppler measurement performed in the past, or may be a parameter provided by an ideal Doppler waveform.

A waveform evaluation section setting unit 34 sets a time section during which evaluation of the current Doppler waveform is performed. When the organism signal measuring device 28 obtains an electrocardiographic waveform, for example, based on this electrocardiographic waveform, a section from an R wave to the next R wave can be regarded as one pulsation cycle of the heart, and this section can be set as a waveform evaluation section. In this case, the Doppler waveform is extracted for each pulsation cycle of the heart and evaluated. Alternatively, a section corresponding to a desired time period from an R wave can be set as a waveform evaluation section. This section may be set as desired, or set in accordance with an item which is subjected to Doppler measurement. For example, when a blood flow velocity in the cardiac systole phase is measured, it is sufficient for evaluation of the Doppler waveform to be performed only in the cardiac systole phase. In such a case, based on the electrocardiographic waveform, the cardiac systole phase is set to the waveform evaluation section.

A Doppler waveform evaluation unit 36 evaluates the current Doppler waveform. Evaluation of the current Doppler waveform is performed by comparing the current Doppler waveform and the suitable waveform information 32 in a section set by the waveform evaluation section setting unit 34. Because a plurality of suitable waveform information items 32 are stored in accordance with the measurement positions, examinees, and the like, as described above, the Doppler waveform evaluation unit 36 selects the suitable waveform information 32 which is appropriate in accordance with the measurement item and the like set by the user. The suitable waveform information 32 selected by the Doppler waveform evaluation unit 36 is a suitable Doppler waveform at a position where the user wishes to perform Doppler measurement, which will be hereinafter referred to as a target measurement position, or a parameter of such a Doppler waveform. It should be noted that the target measurement position does not refer to a single position, but refers to a certain range of positions. In the present embodiment, a range of measurement positions from which Doppler waveforms having Doppler evaluation values, which will be described below, of threshold values or greater can be obtained is considered to be a target measurement position.

The suitable waveform information 32 in various patterns is stored for the same measurement item. As the suitable waveform information 32 corresponding to a measurement item “left ventricle blood inflow”, for example, a group of reference waveforms according to various lesions is stored. The Doppler waveform evaluation unit 36 therefore performs comparison evaluation between the reference waveforms in the group with the current Doppler waveform and specifies, from among the group of reference waveforms, a waveform which is most similar to the Doppler waveform, and then performs evaluation between the current waveform and the specified reference waveform.

The Doppler waveform evaluation unit 36 evaluates the current Doppler waveform to thereby calculate a Doppler evaluation value of the current Doppler waveform. The Doppler evaluation value is an index indicating the degree of suitableness of the current Doppler waveform as a Doppler waveform in the target measurement position, and is calculated based on the degree of correspondence obtained by comparison between the current Doppler waveform and the suitable waveform information 32. The Doppler evaluation value may be represented by a percentage, and is 100 when the current Doppler waveform and the suitable waveform information 32 completely correspond to each other, and is lowered as the degree of correspondence decreases, for example. The Doppler evaluation value which is calculated is transmitted to the display controller 24 and displayed on the display unit 26.

The current Doppler waveform and the suitable waveform information 32 can be compared to each other using various methods. When the reference waveform is stored as the suitable waveform information 32, a shape of the current Doppler waveform and a shape of the reference waveform are compared to each other according to a correlation operation using a cross-correlation function.

When the waveform parameter is stored as the suitable waveform information 32, a parameter corresponding to the suitable waveform information 32 is extracted from the current Doppler waveform, and the corresponding parameters are compared to each other. When the waveform parameters, such as a wave height, a waveform thickness, a noise amount, and attenuation time, are stored as the waveform information 32, the Doppler waveform evaluation unit 36 extracts the wave height, waveform thickness, noise amount, and attenuation time of the current Doppler waveform, and compares each of the parameters to the suitable waveform information 32.

The Doppler waveform evaluation unit 36, based on the calculated Doppler evaluation value, determines whether or not the current Doppler waveform is a suitable waveform. More specifically, when the calculated Doppler evaluation value is equal to or greater than a threshold value which is set, the Doppler waveform evaluation unit 36 determines that the current Doppler waveform is a suitable waveform, whereas when the Doppler evaluation value is less than the threshold value, the Doppler waveform evaluation unit 36 determines that the current Doppler waveform is not a suitable waveform. The threshold value may be modified by the user. The higher the threshold value, the stricter the condition under which the current Doppler waveform is determined as a suitable waveform becomes, that is, the narrower the range of the target measurement positions. Accordingly, when it is desired to set the Doppler measurement position more precisely, the threshold value can be set to a high value.

Because the suitable waveform information 32 is a suitable Doppler waveform or waveform parameter in the target measurement position, a low degree of correspondence between the current Doppler waveform and the suitable waveform information 32, that is, the Doppler evaluation value of the current Doppler waveform which is lower than the threshold value, indicates that the current Doppler measurement position is not set in the target measurement position. Accordingly, in such a case, it is possible to set the current Doppler measurement position to the target measurement position.

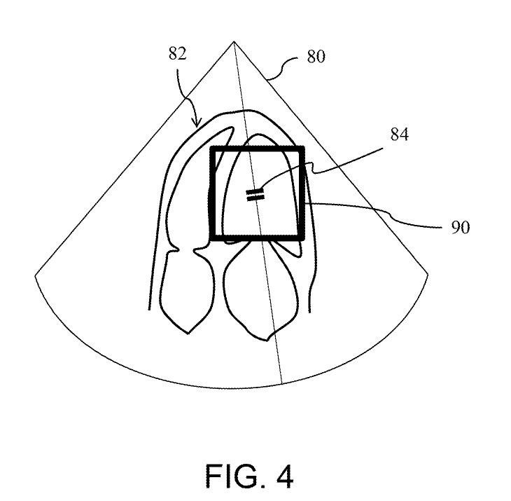

When the Doppler evaluation value which is calculated by the Doppler waveform evaluation unit 36 is smaller than the threshold value, the Doppler measurement position shifting unit 38 shifts the Doppler measurement position to the target measurement position. A new Doppler measurement position can be set based on various standards. A method of setting a new Doppler measurement position will be described below with reference to FIG. 4 through FIG. 6.

Once a new Doppler measurement position is set by the Doppler measurement position shifting unit 38, the Doppler waveform generating unit 22, based on a received signal obtained by Doppler measurement performed in the new Doppler measurement position, generates a Doppler waveform. The Doppler waveform evaluation unit 36 then performs further evaluation of the Doppler waveform. When the Doppler evaluation values is smaller than the threshold value in this further waveform evaluation, the Doppler measurement position shifting unit 38 further shifts the Doppler measurement position. In this manner, the Doppler measurement position shifting unit 38 continues to shift the Doppler measurement position until the Doppler evaluation value becomes equal to or greater than the threshold value, thereby shifting the Doppler measurement position to a position where the Doppler evaluation value is equal to or greater than the threshold value, that is, to the target measurement position.

The warning generating unit 40, when the Doppler evaluation value calculated by the Doppler waveform evaluation unit 36 is equal to or smaller than the threshold value, generates a warning to notify the user of that fact. A warning may be displayed on the display unit 26 or issued by sound or light, or of course may be a combination thereof. The threshold value at which a warning is issued need not be the same as the threshold value at which the Doppler measurement position shifting unit 38 shifts the Doppler measurement position. If the Doppler measurement position is gradually displaced from the target measurement position due to vibrations of the user's hand or the like, for example, by setting the threshold value at which a warning is to be issued to a value higher than the threshold value for shifting the Doppler measurement position, for example, a warning can be issued before the Doppler measurement position is shifted, to notify the user that the Doppler measurement is being displaced, and encourage the user to return the Doppler measurement position to the target measurement position.

The input unit 42 is an interface for performing various operations, and may be an input device such as a keyboard, a track ball, a switch, or a dial. Further, voice input may be allowed. The input unit 42 is used for setting the Doppler measurement position, the cross section type, and the measurement item with respect to which the Doppler measurement is performed, and the like.

A controller 44 is a CPU, for example, and controls the whole system and also operates to perform control based on an instruction input through the input unit 42 by the user.

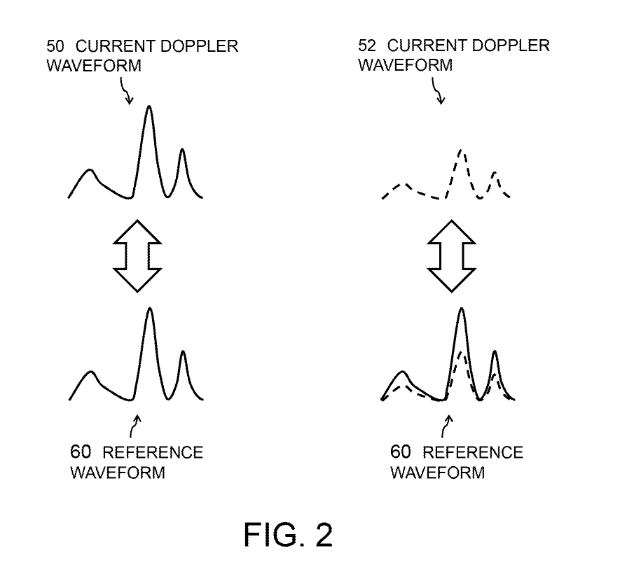

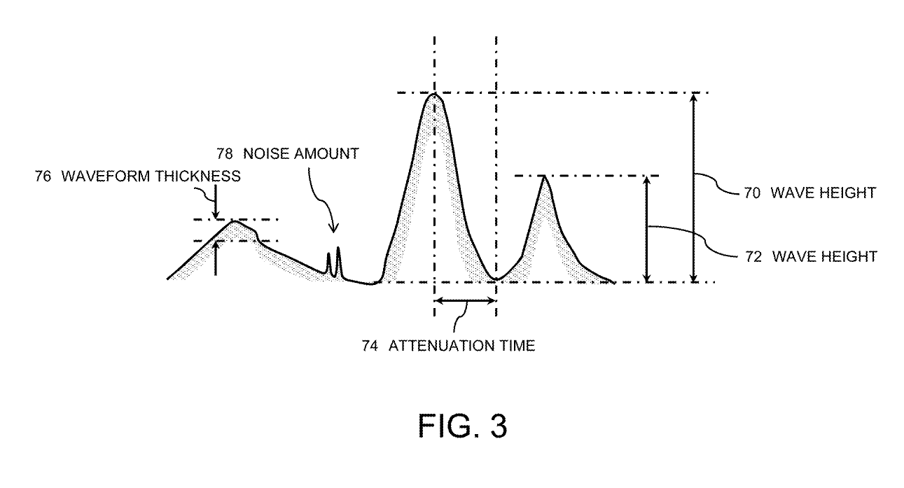

The ultrasonic diagnostic device according to the present embodiment is configured as described above. Among the elements illustrated in FIG. 1, the transmitter/receiver unit 12, the image forming unit 14, the Doppler measurement position setting unit 20, the Doppler waveform generating unit 22, the display controller 24, the waveform evaluation section setting unit 34, the Doppler waveform evaluation unit 36, and the Doppler measurement position shifting unit 38 can be implemented using hardware such as an electronic circuit and a processor, and a device such as a memory may be used for the implementation as required. The functions corresponding to the elements described above may also be implemented by cooperation of hardware, such as a CPU, a processor, and a memory, and software or a software program which regulates the operation of the CPU and the processor. Processing of the Doppler waveform evaluation unit 36 will be described hereinafter with reference to FIG. 1, FIG. 2, and FIG. 3.

FIG. 2 is a conceptual diagram illustrating comparison between the current Doppler waveform and the suitable waveform information. FIG. 2 illustrates comparison between the shapes of the current Doppler waveforms 50 and 52 generated by the Doppler waveform generating unit 22 and the shapes of the reference waveforms 60 based on a correlation operation using a cross-correlation function. The current Doppler waveforms 50 and 52, and the reference waveforms 60 are Doppler waveforms corresponding to one pulsation cycle of the heart and have been extracted from the Doppler waveforms and the reference waveform generated by the Doppler waveform generating unit 22, based on the waveform evaluation section set by the waveform evaluation section setting unit 34.

As described above, the current Doppler waveform has a waveform thickness which varies depending on each time point, for example, and therefore has a complicated shape. Accordingly, in comparing the current Doppler waveforms 50 and 52 with the reference waveforms 60, only the waveform outer shapes may be compared. The waveform outer shape may be a line formed by connecting a point with the highest luminance in each time point, that is, a line connecting values of flow velocity components which are measured most frequently, or may be a line connecting the highest flow velocity value measured at each time point.

With the comparison of the waveform outer shapes only, it is not possible to detect a difference in variations of the blood flow distribution at each time between the two waveforms which are being compared. However, comparison of wave heights and attenuation times is possible by the comparison of the waveform outer shapes only, and the comparison of the waveform outer shapes only is often sufficient in terms of detection of a displacement of the Doppler measurement position. With the comparison of only the waveform outer shapes, it is possible to reduce the operation amount in the comparison processing, which leads to a reduction in time required for the comparison. Of course, waveform comparison in consideration of the waveform thickness may also be performed.

In the example illustrated in FIG. 2, as the current Doppler waveform 50 has a shape which is substantially the same as that of the reference waveform 60, a numeral value close to 100 is calculated as a Doppler evaluation value of the current Doppler waveform 50. However, as the shape of the current Doppler waveform 52 is different from the shape of the reference waveform 60, a significantly low value, e.g., 20, is calculated as a Doppler evaluation value of the current Doppler waveform 52. If the threshold value of the Doppler evaluation value which is used for determining whether or not the current Doppler waveform is suitable is set to 50, the current Doppler waveform 50 is determined as a suitable Doppler waveform, whereas the current Doppler waveform 52 is not determined as a suitable Doppler waveform. It is therefore possible to determine that the Doppler measurement position where the current Doppler waveform 50 was measured is the target measurement position and the Doppler measurement position where the current Doppler waveform 52 was measured is not the target measurement position.

Further, waveform parameters representing the features of a Doppler waveform may be used as the suitable waveform information. FIG. 3 is a diagram illustrating example waveform parameters, including wave heights 70 and 72, attenuation time 74, a waveform thickness 76, and a noise amount 78. The wave heights 70 and 72 are heights of the Doppler waveforms, and indicate the velocity of the blood flow at the peak in the waveform evaluation section. The attenuation time 74 is a time taken from the peak of the blood flow velocity to nearly 0. The waveform thickness 76 indicates a distribution of the blood flow velocity at each time point, as described above. The greater the waveform thickness 76, the greater the difference between the highest blood flow velocity and the lowest blood flow velocity in the Doppler measurement position at that time point. The noise amount 78 is an amount of noise, that is, an amount of a portion of a Doppler waveform which is not considered to indicate the blood flow velocity. Whether or not a portion is a noise is determined based on the wave height of the noise, for example.

When the waveform parameters are used as the suitable waveform information, these parameters are extracted from the current Doppler waveform, and the suitable waveform information is compared with each parameter for overall judgment, thereby calculating the Doppler evaluation value. The Doppler evaluation value may be calculated after weighting each parameter. When the wave height is a significant waveform parameter, for example, a weight of the wave height can be made large, so that the wave height parameter can have a significant effect on the Doppler evaluation value. Such weighting may be modified by a user or different weighting may be set in accordance with the measurement item.

Processing performed by the Doppler measurement position shifting unit 38 will be described hereinafter with reference to FIG. 1, and FIG. 4 to FIG. 6.

FIG. 4 is a diagram illustrating an example search range based on the current Doppler measurement position. FIG. 4 shows a B-mode image 80 formed by the tomographic image forming unit 16. In the example illustrated in FIG. 4, the target tissue is a heart. The B-mode 80 is a tomographic image of the heart 82, which is the target tissue, and shows cross sections of the left ventricle, left atrium, right ventricle, and right atrium of the heart. A cursor 84 indicates the current Doppler measurement position.

When the Doppler waveform evaluation unit 36 determines that the Doppler evaluation value of the current Doppler waveform is equal to or less than the threshold value, the Doppler measurement position shifting unit 38 determines a rectangle 90 having the current Doppler measurement position at the center thereof as a search range, and sets a Doppler measurement position which has been shifted within the search range which is determined. This is based on the fact that the target measurement position often exists in the vicinity of the current Doppler measurement position. The shape of the search range may be, in addition to the rectangle 90, a circle, sparseness?, and the like. As a method of setting a new Doppler measurement position within the search range, general search methods, such as a brute-force search, random search, and hill climbing, can be applied. Searching for a new Doppler measurement position within the predetermined limited range enables expeditious detection of the target measurement position.

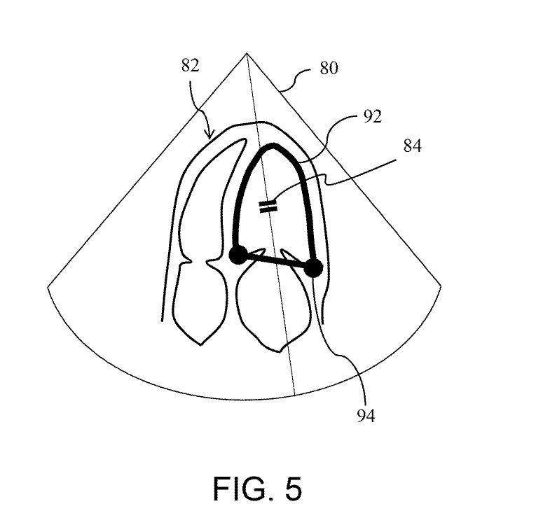

FIG. 5 is a diagram illustrating an example search range based on the reference portion of the target tissue. The Doppler measurement position shifting unit 38 may determine the search range based on the reference portion of the target tissue, such as a specific portion including a contour of the heart cavity and an annulus position, for example. For example, the B-mode image 80 is analyzed to extract the contour of the heart cavity, and the inside of the counter of the heart cavity is set as the search range. Alternatively, the search range may be determined in further consideration of the measurement item. When the “left ventricular blood inflow” is designated as a measurement item, for example, image analysis is performed to specify the left ventricle contour 92, and the inside of the left ventricle contour 92 is set as the search range, as illustrated in FIG. 5. Setting the search range in accordance with the measurement item set by the user and performing searching within this limited range enables not only further expeditious detection of the target measurement position but also prevention of setting the Doppler measurement position to a position which is not desired by the user.

It is further possible to detect a characteristic portion in the target tissue, which is an annulus position 94, for example, and set a predetermined range near this annulus position 94 as the search range. In many cases, the Doppler measurement within the heart is performed so as to measure the blood flow velocity in an inter-valve portion. In these cases, by determining the search range based on the annulus position 94 which is a characteristic portion closer to the inter-valve portion, which is the target measurement position, the search range can be determined at a more accurate position.

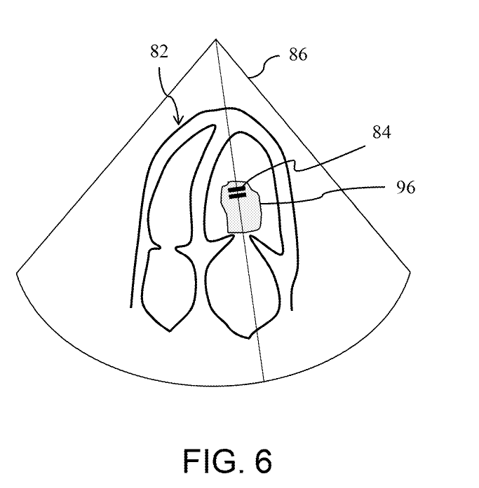

FIG. 6 is a diagram illustrating a search range based on the blood flow information. The Doppler measurement position shifting unit 38 may determine the search range based on the color Doppler image formed by the color Doppler image forming unit 18. FIG. 6 illustrates a CFM image 86 formed of the B-mode image 80 and a color Doppler image superposed on the B-mode image 80. The CFM image 86 shows a velocity distribution 96 of blood flow including an antegrade flow colored in red and a retrograde flow colored in blue, with the flow velocity being indicated by its lightness. The Doppler measurement position shifting unit 38 sets the colored range in the CFM image 86, that is, the range in which the blood flow velocity distribution 96 is shown, as a search range. This setting is made based on the fact that the Doppler measurement is often performed for measuring the blood flow velocity and, in this case, it is highly likely that the target measurement position is a position where the blood flow is present, and particularly a position where the blood flow velocity is high.

Referring to display examples of the display unit 26, specific processing according to the present embodiment will be described.

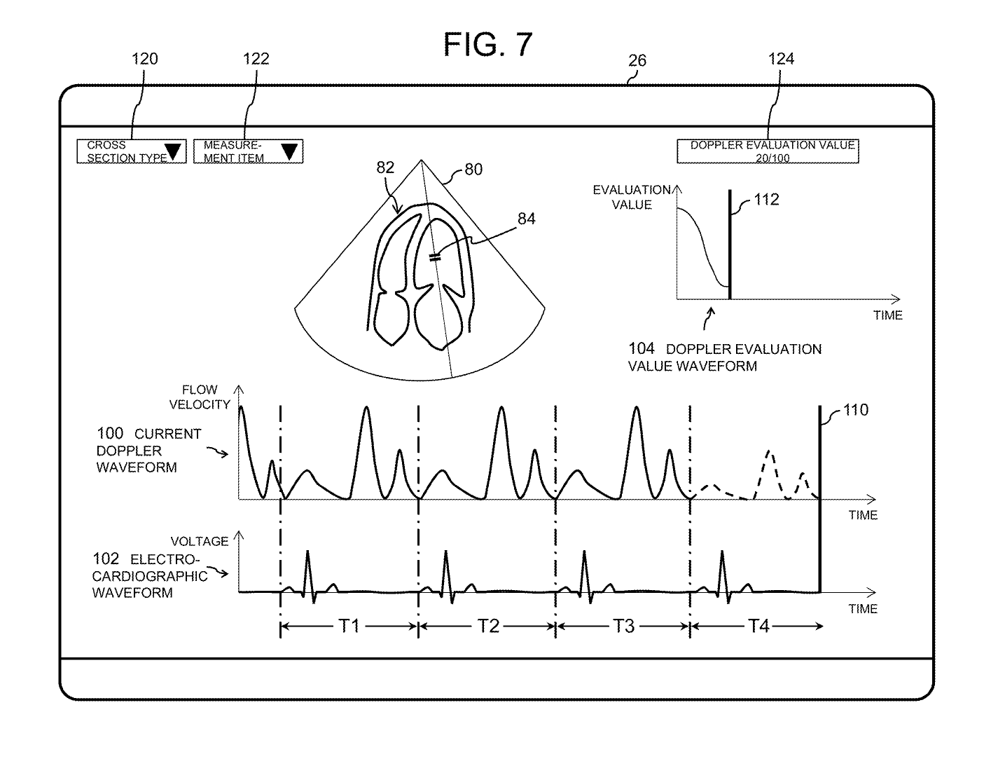

FIG. 7 is a diagram showing the Doppler measurement position having been displaced from the target measurement position. The content of FIG. 7 will be described with reference to FIG. 1. FIG. 7 illustrates a screen displayed in the display unit 26. In the center of the upper portion on the screen, the B-mode image 80 formed by the tomographic image forming unit 16 is shown, in which the target tissue is the heart 82. As the B-mode image 80 is frozen, or is placed in a still image state, once the Doppler measurement has started, during the Doppler measurement, the B-mode image 80 shows a tomographic image of the heart 82 immediately before the start of the Doppler measurement. The B-mode image 80 includes therein the cursor 84 indicating the current Doppler measurement position.

The screen shows, in the lower portion, a current Doppler waveform 100 generated by the Doppler waveform generating unit 22 and an electrocardiographic waveform 102 measured by the organism signal measuring device 28. The current Doppler waveform 100 is a waveform showing the blood flow velocity in the Doppler measurement position indicated by the cursor 84. In a graph including the current Doppler waveform 100, the horizontal axis indicates time and the vertical axis indicates a blood flow velocity. The electrocardiographic waveform 102 is a waveform electrically showing the motion of the heart 82, and is generated based on an organism signal obtained by the organism signal measuring device 28. In a graph including the electrocardiographic waveform 102, the horizontal axis indicates time, and the vertical axis indicates a voltage. The graphs of the current Doppler waveform 100 and the electrocardiographic waveform 102 show a current time bar 110 indicating the current time. The electrocardiographic waveform 102 allows the user to recognize a relationship between the Doppler waveform 100 and the time phase in the pulsation cycle of the heart 82.

The screen further shows, on the right side in the upper portion of the screen, a Doppler evaluation value waveform 104 indicating a transition of the Doppler evaluation value with time, and a Doppler evaluation value window 124 indicating the current Doppler evaluation value. The value indicated by the Doppler evaluation value window 124 is a Doppler evaluation value at the current time, which is calculated based on the comparison between the current Doppler waveform and the suitable waveform information 32, as described above. The Doppler evaluation value is expressed as a percentage, which is higher as the waveform is more suitable. In a graph including the Doppler evaluation value waveform 104, the horizontal axis indicates time and the vertical axis indicates the Doppler evaluation value. The graph including the Doppler evaluation value waveform 104 also shows thereon a current time bar 112 indicating the current time. With the Doppler evaluation value waveform 104, the user can recognize a transition of the Doppler evaluation value with time.

The screen also shows, on the left side of the upper portion, a cross section type window 120 and a measurement item window 122. The cross section window 120 indicates a cross section type of the B-mode image 80. The cross section type may be input by the user through the input unit 42, or may be automatically determined by analysis of the B-mode image, which is performed by the tomographic image forming unit 16. The cross section type of the B-mode image 80 illustrated in FIG. 7 is an “apical 4-chamber view”. The measurement item window 122 indicates a target of the Doppler measurement. The measurement item is input by the user through the input unit 42. Based on the cross section type and the measurement item, the suitable waveform information for use in the comparison with the current Doppler waveform for calculating the Doppler evaluation value is selected.

Each of the current Doppler waveform 100 and the electrocardiographic waveform 102 is divided into a plurality of time sections T1, T2, T3, and T4, which correspond to the waveform evaluation sections set by the waveform evaluation section setting unit 34, respectively. While the dashed and single-dotted lines and the like indicating each waveform evaluation section are not actually shown, these lines may be actually displayed such that the user can recognize each waveform evaluation section. In the example illustrated in FIG. 7, a waveform evaluation section is set to one heartbeat of the heart.

In the example illustrated in FIG. 7, while, in the waveform evaluation sections T1, T2, and T3, the Doppler measurement position is set to the target measurement position, in the waveform evaluation section T4, the Doppler measurement position is displaced from the target measurement position due to vibrations of hands, for example. As indicated by the current Doppler waveform 100, the Doppler waveform in T4 is different from the Doppler waveform in T1 to T3 in that the Doppler waveform in T4 has a lower wave height, for example. As shown in the Doppler evaluation value window 124, the Doppler evaluation value at the current time, which is included in the waveform evaluation section T4, is 20. In the present embodiment, the threshold value of the Doppler evaluation value which is a criteria for determining whether or not the Doppler waveform is suitable is set to 50, and therefore the Doppler waveform evaluation unit 36 determines that the current Doppler waveform in the waveform evaluation section T4 is not a suitable Doppler waveform.

In order to allow the user to recognize that the Doppler waveform is not determined as a suitable Doppler waveform, the current Doppler waveform in the waveform evaluation section T4 is displayed in a color which is different from the color of the current Doppler waveform in a section in which the Doppler waveform is determined to be suitable, for example, such that these waveforms can be distinguished from each other. Also, when the Doppler waveform evaluation value is equal to or smaller than the threshold value, warning message generated by the warning generating unit 40 is also displayed.

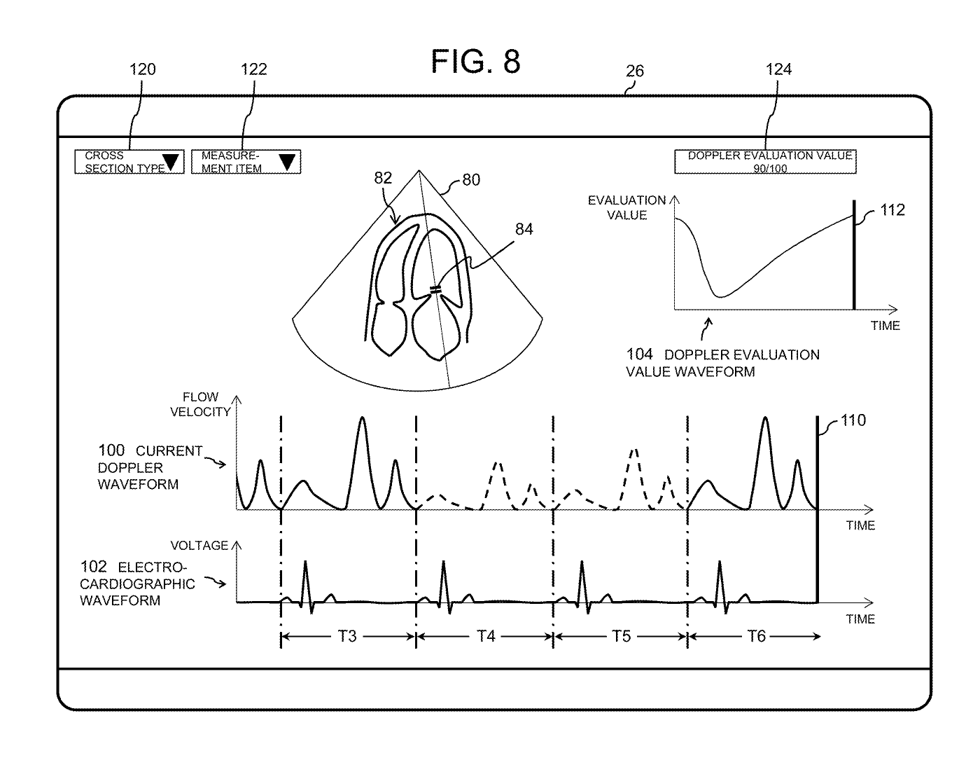

FIG. 8 is a diagram illustrating the Doppler measurement position having been reset to the target measurement position. The content of FIG. 8 will be described with reference to FIG. 1. FIG. 8 illustrates a display content of the display unit 26 after elapse of a predetermined time from the state illustrated in FIG. 7. FIG. 7 illustrates the waveform evaluation sections T1 to T4, whereas FIG. 8 illustrates waveform evaluation sections T3 to T6. The waveform evaluation sections T3 and T4 in FIG. 7 and the waveform evaluation section T3 and T4 in FIG. 8 are the same sections.

As described above, the Doppler waveform evaluation unit 36 determines that the Doppler evaluation value is equal to or smaller than the threshold value in the waveform evaluation section T4, that is, that the Doppler measurement position has been displaced from the target measurement position in the waveform evaluation section T4. In response, the Doppler measurement position shifting unit 38 performs processing for shifting the Doppler measurement position after elapse of the waveform evaluation section T4. Consequently, in the waveform evaluation section T5, the current Doppler waveform is generated at the Doppler measurement position which is shifted.

In the example illustrated in FIG. 8, the Doppler evaluation value of the current Doppler waveform in the waveform evaluation section T5 is also determined as being equal to or less than the threshold value. In other words, the Doppler measurement position which has been shifted is not the target measurement position, either. The Doppler measurement position shifting unit 38 therefore further shifts the Doppler measurement position after completion of the waveform evaluation section T5. Consequently, in the waveform evaluation section T6, the current Doppler waveform is generated at the Doppler measurement position which is further shifted. The Doppler evaluation value of the current Doppler waveform in the waveform evaluation section T6 is 90, as shown in the Doppler evaluation value window 124, which is greater than the threshold value, 50. As such, the Doppler measurement position which has been further shifted is the target measurement position. As described above, in the example illustrated in FIG. 8, after performing the Doppler measurement position shifting processing twice, the Doppler measurement position is shifted to the target measurement position. The Doppler evaluation value waveform 104 in FIG. 8 shows a gradual correction of the Doppler evaluation value.

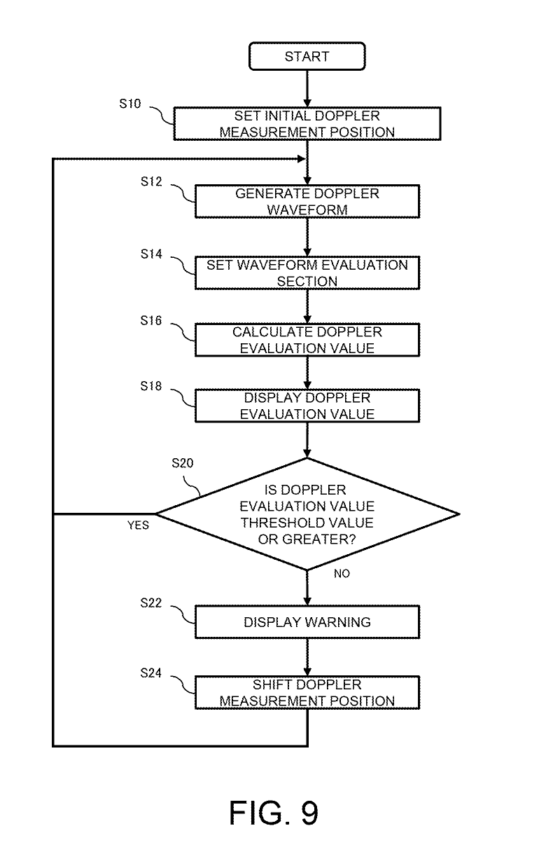

Hereinafter, the processing performed by the ultrasonic diagnostic device according to the present embodiment will be described. FIG. 9 is a flowchart showing the operation of the ultrasonic diagnostic device according to the present embodiment. The content of the flowchart in FIG. 9 will be described with reference to FIG. 1.

In step S10, the Doppler measurement position setting unit 20 sets an initial Doppler measurement position. As described above, the user moves a cursor on the B-mode image displayed on the display unit 26 using a trackball and the like in the input unit 42 to thereby designate the initial Doppler measurement position, and the Doppler measurement position setting unit 20 sets the designated position as the initial Doppler measurement position.

In step S12, the Doppler waveform generating unit 22 generates, based on an ultrasound signal transmitted to and received from the Doppler measurement position, a current Doppler waveform indicating the blood flow velocity in the Doppler measurement position.

In step S14, the waveform evaluation section setting unit 34 sets, based on an electrocardiographic waveform obtained by the organism signal measuring device 28, a waveform evaluation section which is a target section for evaluating the current Doppler waveform. In the present embodiment, one heartbeat period of the heart is set as the waveform evaluation section based on the electrocardiographic waveform.

In step S16, the Doppler waveform evaluation unit 36 evaluates the current Doppler waveform generated in step S12 and calculates a Doppler evaluation value. Evaluation of the current Doppler waveform is performed with respect to the waveform evaluation section set in step S14. Further, calculation of the current Doppler evaluation value is performed by comparing the suitable waveform information 32 stored in the storage unit 30 with the current Doppler waveform, as described above.

In step S18, the display controller 24 receives the Doppler evaluation value which is calculated from the Doppler waveform evaluation unit 36, and displays the Doppler evaluation value on the display unit 26.

In step S20, the Doppler waveform evaluation unit 36 determines whether or not the Doppler evaluation value is equal to or greater than the threshold value. If the Doppler evaluation value is equal to or greater than the threshold value, which means that the current Doppler measurement position is the target measurement position, the process returns to step S12, and continues Doppler measurement in the current Doppler measurement position. If the Doppler evaluation value is smaller than the threshold value, on the other hand, as it can be determined that the current Doppler measurement position has been displaced from the target measurement position, output of warning and shift of the Doppler measurement is performed in the following steps S22 and S24, respectively.

Specifically, in step S22, the warning generating unit 40, triggered by the Doppler evaluation value which is smaller than the threshold value, outputs a warning message to the display controller 24, which then causes the display unit 26 to display the warning message.

In step S24, the Doppler measurement position shifting unit 38, triggered by the Doppler evaluation value which is smaller than the predetermined value, shifts the current Doppler measurement. After completion of the processing for shifting the Doppler measurement position, the process returns to step S12 and similar processing is repeated. Whether or not the shifted Doppler measurement position is the target measurement position is determined in step S20. As described above, so long as the Doppler evaluation value is smaller than the threshold value, that is, so long as the Doppler measurement position is set to a position other than the target measurement position, the Doppler position is continuously shifted. With this processing, the Doppler measurement position which has been displaced from the target measurement position is set back to the target measurement position.

According to the present embodiment, the Doppler measurement position is searched and corrected such that a proper Doppler waveform can always be obtained even when the Doppler measurement position has been displaced from the target measurement position due to a movement of the body or vibration of a hand, for example. Thus, time and labor required for the user to reset the Doppler measurement position can be alleviated. This structure not only results in an increase in the examination efficiency but also allows anyone to shift the Doppler measurement position so as to obtain a proper Doppler waveform, so that variations in the measurement results among different users can be reduced.

### REFERENCE SIGNS LIST

10 probe, 12 transmitter/receiver unit, 14 image forming unit, 16 tomographic image forming unit, 18 color Doppler image forming unit, 20 Doppler measurement position setting unit, 22 Doppler waveform generating unit, 24 display controller, 26 display unit, 28 organism signal measurement device, 30 storage unit, 32 suitable waveform information, 34 waveform evaluation section setting unit, 36 Doppler waveform evaluation unit, 38 Doppler measurement position shifting unit, 40 warning generating unit, 42 input unit, 44 controller, 50, 52 current Doppler waveform, 60 reference waveform, 70, 72 wave height, 74 attenuation time, 76 waveform thickness, 78 noises amount, 80 B-mode image, 82 heart, 84 cursor, 86 CFM image, 90 rectangle, 92 left ventricle contour, 94 annulus position, 96 velocity distribution, 100 current Doppler waveform, 102 electrocardiographic waveform, 104 Doppler evaluation value waveform, 110, 112 current time bar, 120 cross section type window, 122 measurement item window, 124 Doppler evaluation value window.

1. An ultrasonic diagnostic device comprising:
a Doppler waveform generator configured to generate, based on a received signal obtained by transmitting and receiving ultrasound waves to and from a Doppler measurement position set for measuring a motion concerning a target tissue in a target measurement position within the target tissue, a Doppler waveform indicating the motion in the Doppler measurement position;
a waveform evaluator configured to perform waveform evaluation of the Doppler waveform in accordance with a measurement item for measurement of the motion, to thereby evaluate whether or not the Doppler waveform satisfies a suitable condition required for a suitable Doppler waveform in the target measurement position, as a result of the measurement concerning the measurement item; and
a Doppler measurement position shifter configured to shift the Doppler measurement position when the Doppler waveform does not satisfy the suitable condition.

2. The ultrasonic diagnostic device according to claim 1, wherein
the Doppler measurement position shifter shifts the Doppler measurement position to a measurement position where a Doppler waveform which satisfies the suitable condition can be obtained.

3. An ultrasonic diagnostic device comprising:
a Doppler waveform generator configured to generate, based on a received signal obtained by transmitting and receiving ultrasound waves to and from a Doppler measurement position set for measuring a motion concerning a target tissue in a target measurement position within the target tissue, a Doppler waveform indicating the motion in the Doppler measurement position;
a waveform evaluator configured to perform waveform evaluation of the Doppler waveform, to thereby evaluate whether or not the Doppler waveform satisfies a suitable condition required for a suitable Doppler waveform in the target measurement position; and
a Doppler measurement position shifter configured to shift the Doppler measurement position when the Doppler waveform does not satisfy the suitable condition, wherein
the suitable condition is a condition concerning a degree of correspondence between a current Doppler waveform generated by the Doppler waveform generator and a reference waveform to be compared therewith, and
the waveform evaluator determines whether or not the suitable condition is satisfied based on the degree of correspondence.

4. The ultrasonic diagnostic device according to claim 3, wherein
the waveform evaluator calculates, as the degree of correspondence, a degree of correspondence of waveform shapes, based on a shape of the reference waveform and a shape of the current Doppler waveform.

5. The ultrasonic diagnostic device according to claim 3, wherein
the waveform evaluator calculates, as the degree of correspondence, a degree of correspondence of parameters, based on a parameter representing a feature of the reference waveform and a parameter representing a feature of the current Doppler waveform.

6. The ultrasonic diagnostic device according to claim 3, wherein
the waveform evaluator calculates an evaluation value based on the degree of correspondence and indicates the evaluation value on a display unit.

7. The ultrasonic diagnostic device comprising:
a Doppler waveform generator configured to generate, based on a received signal obtained by transmitting and receiving ultrasound waves to and from a Doppler measurement position set for measuring a motion concerning a target tissue in a target measurement position within the target tissue, a Doppler waveform indicating the motion in the Doppler measurement position;
a waveform evaluator configured to perform waveform evaluation of the Doppler waveform, to thereby evaluate whether or not the Doppler waveform satisfies a suitable condition required for a suitable Doppler waveform in the target measurement position; and
a Doppler measurement position shifter configured to shift the Doppler measurement position when the Doppler waveform does not satisfy the suitable condition, wherein
the waveform evaluator determines whether or not the Doppler waveform satisfies the suitable condition in a predetermined time phase section during a pulsation cycle of the target tissue.

8. The ultrasonic diagnostic device according to claim 7, wherein
the target tissue is a heart, and
the waveform evaluator determines the predetermined time phase section based on an electrocardiographic waveform of the heart.

9. The ultrasonic diagnostic device according to claim 1, wherein
the Doppler measurement position shifter searches for a new measurement position within a search range determined based on the Doppler measurement position.
